(19) United States
(12) Patent Application Publication
(10) Pub. No.: US 2026/0060814 A1
(43) Pub. Date: Mar. 5, 2026

(54) SURGICAL TOOL

(71) Applicant: Conor McKinnis, Johnson city, TN (US)

(72) Inventor: Conor McKinnis, Johnson city, TN (US)

(21) Appl. No.: 19/317,036

(22) Filed: Sep. 2, 2025

Related U.S. Application Data
(60) Provisional application No. 63/689,600, filed on Aug. 30, 2024.

(51) Int. Cl. A61F 2/46

(52) U.S. Cl. CPC A61F 2/461

(57) ABSTRACT

The present apparatus is a tool that holds and positions the tibial insert to improve the process of inserting the insert during a knee replacement operation. The apparatus comprises an arcuate convex shape that receives and holds the insert in place. The apparatus can be attached to a handle to provide greater ease of use, more reliable handling, and easier placement. The apparatus is made to have a pressure fit on the insert to firmly hold it in place, while still allowing it to be pulled off after the insert has been placed.

This patent application claims priority to U.S. Provisional Patent Application 65/689,600 filed on Aug. 30, 2025, which is incorporated by reference herein in its entirety.

### BACKGROUND OF THE SYSTEM

Knee replacement surgery is a well-known orthopedic treatment for injured or damaged knees. There are a number of companies that provide knee replacement hardware. One prior art knee replacement system is manufactured by DePuy and includes something referred to as the Attune Rotating Platform. This is a platform implanted on the tibia to form a flat platform that can then receive a poly tibial insert that can rotate axially to provide more natural knee movement and to reduce stress during normal knee use.

A disadvantage of the current procedures used for the insertion of the poly tibial insert is that it is done manually. Often metal tools are used to help seat the insert. The manual insertion is slow and can take 30 minutes or more for the operation. In addition, the metal tools used to help seat the poly insert can scratch or damage the insert. Often a surgery will require two inserts to be available in case one is damaged. In addition, the prior art insertion technique can actually lead to bone fractures of the natural portions of the knee. This results in post-operative problems and diminished quality of life.

### SUMMARY

The present apparatus is a tool that holds and positions the tibial insert to improve the process of inserting the insert during a knee replacement operation. The apparatus comprises an arcuate convex shape that receives and holds the insert in place. The apparatus can be attached to a handle to provide greater ease of use, more reliable handling, and easier placement. The apparatus is made to have a pressure fit on the insert to firmly hold it in place, while still allowing it to be pulled off after the insert has been placed.

### BRIEF DESCRIPTION OF THE DRAWINGS

FIG. 1 is an example of a tibial platform and poly insert in an embodiment.

FIG. 2 is a plan view of an embodiment of the apparatus.

FIG. 3 is a perspective view of the apparatus attached to a handle.

FIG. 4 illustrates the apparatus holding a poly insert.

### DETAILED DESCRIPTION OF THE SYSTEM

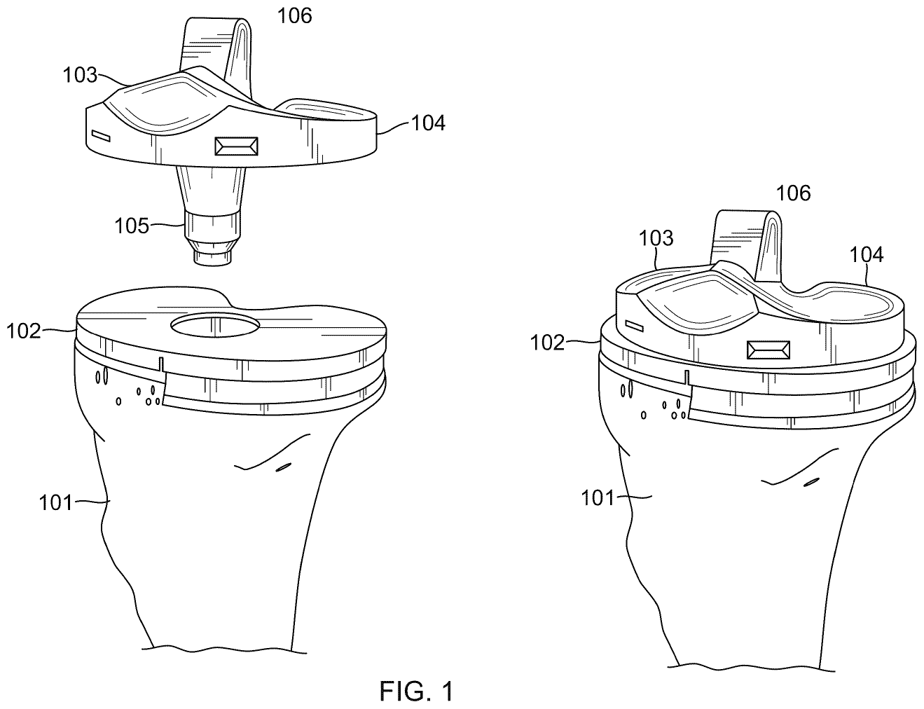

FIG. 1 illustrates a tibial platform and poly insert in an embodiment. During a knee replacement surgery, a tibial platform 102 is inserted into a tibia 101. The tibial platform 102 includes an opening to receive a lower shaft 105 of insert 103. The insert 103 includes a seat 104 which rests on tibial platform 102 when inserted during the procedure. The insert includes an upper shaft 106.

In current operation, the lower shaft 105 insert 103 is inserted manually into an opening in the tibial platform 102. The insert is then forced into place by applying pressure to the seat via a tool that is struck to fully seat the insert 103. The tool in some cases is a hollow metal tube that is placed over the upper shaft 106 and tapped with a hammer to apply downward pressure to the insert 103. The lower shaft 105 of the insert 103 is tapered to create a pressure fit with the opening in the tibial platform 102 when fully seated.

After the insert 103 is seated in the tibial platform, the femur must be placed on the upper shaft 106. This process cause a great deal of stress on the implant, resulting in lateral stress on the insert 103, the lower shaft 105, and ultimately on the tibia itself. Because the knee is bent, and the upper shaft 106 is to be inserted into the femur, it is not possible to have the femur go straight onto the upper shaft 106. It is inserted at an angle and then pushed in until it is seated. Only then can the leg be in a fully straight position.

The present tool allows the pressure during the process to be controlled by the doctor, pushing in the opposite direction of the lateral force, reducing the forces applied by the lower shaft 105 on the tibia. The unique shape of the tool allows it to evenly apply counterforce without concentrating force in any one location, reducing the stress on the insert and on the tibia and femur.

A disadvantage of the manual placement of the insert 103 is that the current tools to force the insert 103 in place can damage the insert and/or cause stress or fractures in the tibia 101. Some surgeons insist on two inserts 103 being available because of the meaningful risk of damage to the insert during placement. The second insert is used to replace a damaged insert when necessary. This adds unwanted cost to the procedure. In addition, the manual placement of the insert 103 takes significant time, requiring the patient to be under anaesthesia for a longer period of time.

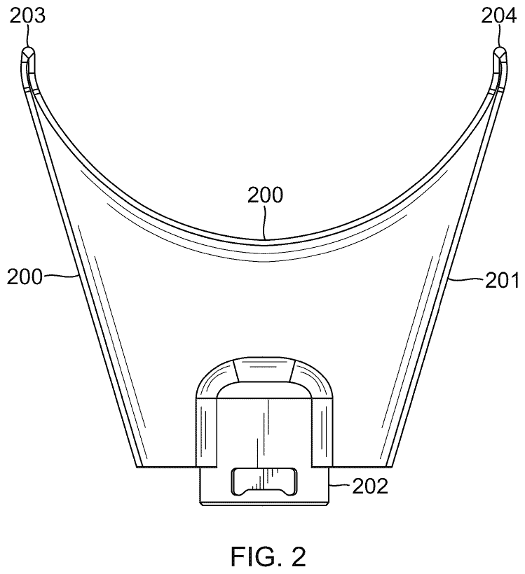

FIG. 2 illustrates a plan view of an embodiment of an apparatus for holding the insert 103. The apparatus 200 comprises a main body 201 and attachment means 202. The main body 201 forms a curved region 205 that ends in tips 203 and 204. The curved region 205 is not a continuous arc but becomes slightly shallower at tip 203 and 204. The tips are pushed around an insert 103 in a pressure fit to hold it in place. The curved region 205 matches an outer curve of the insert 103 so that equal and even pressure is applied on the edges of insert 103 during operation. The attachment 202 is formed so that a handle may be removably coupled and removed from apparatus 200 as desired.

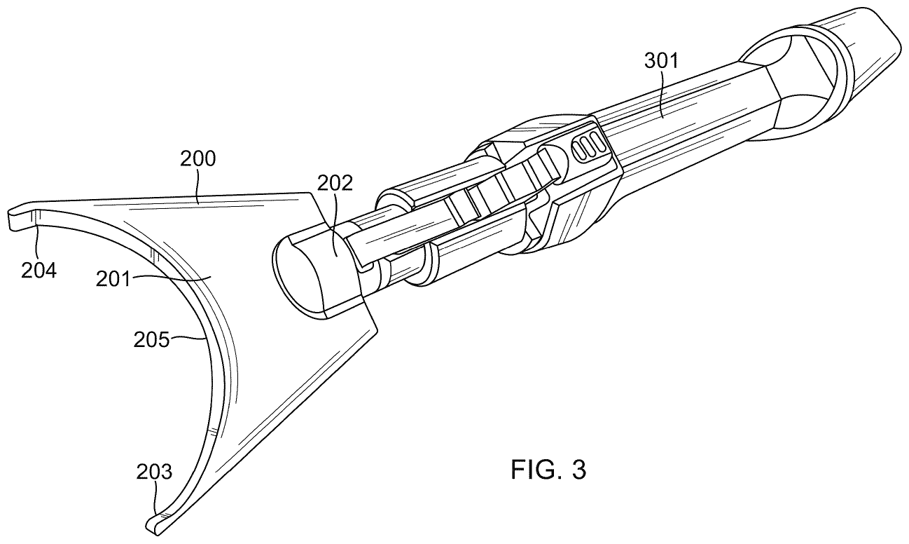

FIG. 3 illustrates a perspective view of an embodiment of the apparatus 200 attached to a handle 301. In one embodiment, the handle is a DePuy impaction handle. This handle includes a latch to engage a tool and a release that allows the handle to be disengaged as desired. In one embodiment, the apparatus includes an attachment 202 designed to engage the impaction handle 301, because the impaction handle can be used with other devices and apparatuses typically used in a knee replacement procedure.

In one embodiment other releasable or non-releaseable handles are used with the apparatus 200. It should be noted that the tool can be used with other tibial inserts without departing from the scope and spirit of the apparatus.

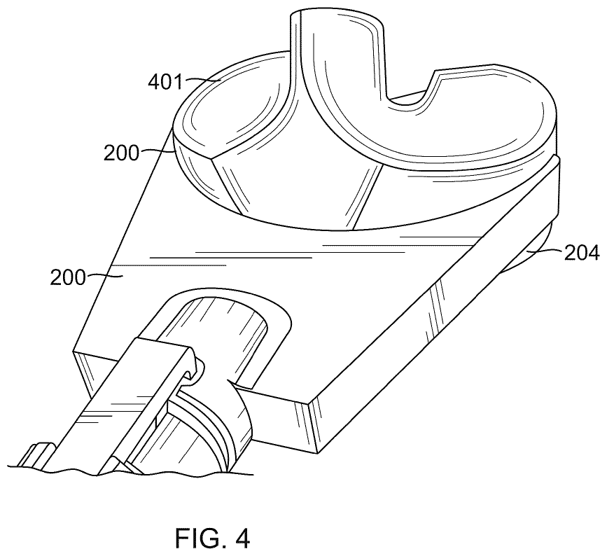

FIG. 4 illustrates the apparatus 200 holding an insert 401. The insert 401 can be inserted into the curved region 205 of the apparatus 200 such that the insert 401 is gripped by tips 203 and 204. The holder 200 can allow the insert to be more easily applied to the tibial platform with greater speed and with less trauma than with the prior art methods.

What is claimed is:

1. An apparatus for positioning a tibial insert during knee replacement surgery, comprising:
a main body having a curved region terminating in opposing tips, the curved region dimensioned to engage an outer surface of the tibial insert; and
an attachment portion configured to couple to a handle;
wherein the opposing tips are adapted to apply substantially even pressure against the tibial insert to hold the tibial insert in a pressure fit during insertion into a tibial platform.

2. The apparatus of claim 1, wherein the curved region is arcuate and convex relative to the tibial insert.

3. The apparatus of claim 1, wherein the attachment portion is configured to releasably engage an impaction handle.

4. The apparatus of claim 1, wherein the opposing tips are shallower than a central portion of the curved region to facilitate gripping of the tibial insert.

5. The apparatus of claim 1, wherein the main body is formed of a polymeric or composite material to reduce risk of scratching the tibial insert.

6. A method of inserting a tibial insert during knee replacement surgery, comprising:
providing an apparatus having a curved region with opposing tips configured to grip the tibial insert;
placing the tibial insert into the curved region such that the opposing tips form a pressure fit with the tibial insert;
attaching a handle to the apparatus; and
seating the tibial insert into a tibial platform while maintaining substantially even pressure on the tibial insert by the curved region of the apparatus.

7. The method of claim 6, wherein attaching the handle comprises releasably coupling the apparatus to an impaction handle.

8. The method of claim 6, further comprising applying counterforce with the apparatus to reduce lateral stress on the tibial insert during insertion.

9. The method of claim 6, wherein the apparatus is removed from the tibial insert after the tibial insert is fully seated.

10. The method of claim 6, wherein the tibial insert comprises a lower shaft dimensioned to form a pressure fit with an opening in the tibial platform.

11. A system for knee replacement surgery, comprising:
a tibial platform configured to be implanted in a tibia, the tibial platform having an opening;
a tibial insert having a lower shaft dimensioned to engage the opening of the tibial platform and a seat configured to rest on the tibial platform; and
an apparatus comprising a curved region terminating in opposing tips, the curved region dimensioned to engage an outer surface of the tibial insert in a pressure fit to hold the tibial insert during insertion into the tibial platform.

12. The system of claim 11, further comprising an attachment portion on the apparatus configured to releasably couple to a surgical handle.

13. The system of claim 11, wherein the curved region is arcuate and convex and exerts substantially even pressure on the tibial insert.

14. The system of claim 11, wherein the tibial insert further comprises an upper shaft configured to engage a femoral component.

15. The system of claim 11, wherein the apparatus is formed of a polymeric material to minimize scratching of the tibial insert during insertion.
